(19) United States
(12) Patent Application Publication
(10) Pub. No.: US 2016/0066848 A1
(43) Pub. Date: Mar. 10, 2016

(54) WEARABLE ENVIRONMENTAL POLLUTION MONITOR COMPUTER APPARATUS, SYSTEMS, AND RELATED METHODS

(71) Applicant: Vision Service Plan, Rancho Cordova, CA (US)

(72) Inventors: Richard Chester Klosinski, JR., Sacramento, CA (US); Meghan Kathleen Murphy, Davis, CA (US); Matthew Allen Workman, Sacramento, CA (US); Jay William Sales, Citrus Heights, CA (US)

(21) Appl. No.: 14/846,401

(22) Filed: Sep. 4, 2015

Related U.S. Application Data
(60) Provisional application No. 62/046,406, filed on Sep. 5, 2014.
(60) Provisional application No. 62/135,612, filed on Mar. 19, 2015.

(51) Int. Cl. A61B 5/00; A61B 5/145; A61B 5/08

(52) U.S. Cl. CPC A61B 5/6803; A61B 5/08; A61B 5/0077; A61B 5/14542; A61B 5/445; A61B 2560/0242

(57) ABSTRACT

A system, according to various embodiments, includes eyewear (or any other suitable wearable device) that includes at least one environmental pollution monitoring sensor (e.g., at least one light pollution, air pollution, radioactive pollution, thermal pollution, and/or noise pollution sensor) that may be used to monitor the presence of environmental pollution adjacent a wearer of the eyewear. The system may further include one or more health monitoring sensors to determine the health effects of the environmental pollution on the wearer. In certain circumstances, the system may alert the user to potentially harmful environmental conditions, or convey preventative healthcare advice to the wearer.

### CROSS-REFERENCE TO RELATED APPLICATIONS

This application claims the benefit of U.S. Provisional Patent Application No. 62/135,612, filed Mar. 19, 2015, entitled “Wearable Environmental Pollution Monitor Computer Apparatus, Systems, and Related Methods,” and U.S. Provisional Patent Application No. 62/046,406, filed Sep. 5, 2014, entitled “Wearable Health Computer Apparatus, Systems, and Related Methods,” the entire disclosures of which are incorporated herein by reference in their entirety.

### BACKGROUND

Being able to detect the amount and type of environmental pollution directly affecting a particular person may be of great importance to that person—especially if they have a medical condition that is aggravated by environmental pollution. Accordingly, there is a need for effective systems and methods for monitoring and detecting localized environmental pollutants.

### SUMMARY OF VARIOUS EMBODIMENTS

A computer-readable medium, according to various embodiments, comprises computer-executable instructions for: (1) receiving environmental pollution data from at least one environmental pollution sensor attached adjacent a wearable device; and (2) receiving health data from at least one health sensor attached adjacent the wearable device. The health data, in various embodiments, indicates at least one aspect of the health of a wearer of the wearable device. In various embodiments, the computer-readable medium further comprises computer-executable instructions for using the environmental pollution data and the health data to determine that a particular type and/or level of environmental pollution, as determined at least in part from the environmental pollution data, is associated with the at least one aspect of the wearer's health. In response to determining that the particular type and/or level of environmental pollution is associated with the at least one aspect of the wearer's health, the system may generate a message indicating the association between the particular type and/or level of environmental pollution and the at least one aspect of the wearer's health.

A computer system, according to various embodiments, comprises at least one computer processor and is configured for receiving environmental pollution data from a plurality of environmental sensors, each of the sensors being attached adjacent a respective particular wearable device (e.g., eyewear) that is being worn by a respective particular user while the user is located within a particular geographical zone. The system uses the pollution data from each of the plurality of environmental sensors to determine a respective level of a particular pollutant at a particular respective location within the particular geographical zone. The system may use the determined respective levels of the particular pollutant at particular respective locations within the particular geographical zone to determine an approximate aggregate level of the particular pollutant within the particular geographical zone.

A computer-implemented method, according to various embodiments, of monitoring environmental pollution adjacent a wearable device, comprises receiving, by at least one processor, environmental pollution data from at least one environmental pollution sensor attached adjacent a wearable device. The method further comprises receiving, by at least one processor, health data from at least one health sensor attached adjacent the wearable device. The health data indicates at least one aspect of the health of a wearer of the wearable device. The method further comprises using, by at least one processor, the environmental pollution data and the health data to determine that a particular type and/or level of environmental pollution, as determined at least in part from the environmental pollution data, is associated with the at least one aspect of the wearer's health. The method further comprises the step of using at least one processor to, in response to determining that the particular type and/or level of environmental pollution is associated with the at least one aspect of the wearer's health, generate a message indicating the association between the particular type and/or level of environmental pollution and the at least one aspect of the wearer's health.

### BRIEF DESCRIPTION OF THE DRAWINGS

Various embodiments of systems and methods for assessing a level of environmental pollution are described below. In the course of this description, reference will be made to the accompanying drawings, which are not necessarily drawn to scale and wherein:

FIG. 1 is a block diagram of an Environmental Pollution Monitoring System in accordance with an embodiment of the present system.

FIG. 2 is a block diagram of the Environmental Monitoring Pollution Server of FIG. 1.

FIG. 3 depicts a flowchart that generally illustrates various steps executed by an Environmental Pollution Monitoring Module according to a particular embodiment.

FIG. 4 is an exemplary wearable health monitoring device for use in the Environmental Pollution Monitoring System of FIG. 1.

### DETAILED DESCRIPTION OF SOME EMBODIMENTS

Various embodiments will now be described more fully hereinafter with reference to the accompanying drawings. It should be understood that the invention may be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

### Overview

A wearable environmental pollution monitoring system, in various embodiments, may, for example, be embodied in any suitable wearable device configured to monitor the physical condition of a wearer and/or the localized level of environmental pollution affecting the wearer. The system may, for example, be embodied as: (1) a pair of eyewear; (2) one or more contact lenses; (3) a wristwatch; (4) a suitable piece of clothing (e.g., a suitable shirt, pair of pants, undergarment, compression sleeve, etc.); (5) footwear; (6) a hat; (7) a helmet; (8) an orthopedic cast; or (9) any other suitable wearable item or other suitable item.

In various embodiments, the system comprises: (1) one or more pollution sensors, which are configured to determine an individual's exposure to one or more environmental pollutants; and/or (2) one or more health sensors to monitor the wearer's reaction to these pollutants. The one or more pollution and/or health sensors may be coupled to the wearable device in any suitable way. For instance, the one or more sensors may be embedded into the wearable device, coupled to the wearable device, and/or operatively coupled to the wearable device.

In various embodiments, the wearable environmental monitoring system may enable the system to determine a level of one or more environmental pollutants, (e.g., air pollutants, light pollutants, noise pollutants, radioactive pollutants, and/or thermal pollutants) using the environmental monitoring system's environmental pollution sensors when the environmental pollution monitoring system is worn by the wearer. The one or more pollution sensors may include, for example, one or more air sampling devices, one or more light pollutant samplers (e.g., one or more devices for measuring light pollutants such as ultraviolet light and blue light), one or more noise pollutant samplers, one or more radioactive pollutant monitors, one or more thermal pollutant samplers, and/or one or more ultraviolet light measurement devices.

In various embodiments, the wearable environmental monitoring system may enable the system to determine the reaction of the wearer to one or more environmental pollutants by using the environmental monitoring system's one or more health sensors. In various embodiments, the one or more health sensors measure a physical reaction of the wearer to various environmental pollutants by monitoring certain characteristics of the wearer including, for example, changes in: pupil size, skin coloration, heart rate, perspiration level, composition of the wearer's perspiration, respiration rate, movement, brainwave activity, and/or any other suitable characteristics. The one or more health sensors may include, for example, one or more heart rate monitors, one or more electrocardiograms (EKGs), one or more electroencephalograms (EEGs), one or more thermometers, one or more transdermal sensors, one or more blood pressure sensors, one or more pulse oximeters, one or more respiration rate monitors, one or more front-facing cameras, one or more eye-facing cameras, one or more communication sensors, or any other suitable one or more sensors.

In various embodiments, the system is configured to associate the wearer's physical reaction with one or more environmental pollutants based, at least in part, on the received health data and environmental pollution signals. For example, if the system senses that the wearer has an otherwise unexplained increase in heart rate or respiratory rate, and the system senses the presence of a particular environmental pollutant, the system may determine that the wearer is having an adverse reaction to the environmental pollutant.

After determining that a wearer is having a possible adverse reaction to a particular pollutant, the system may notify the wearer or a third party (e.g., the wearer's physician) of the possible adverse reaction. In particular embodiments, the system may notify the wearer via the wearable device or through a notification sent to a mobile computing device or other computing device associated with the wearer or other individual. The system may also provide the wearer with one or more suggestions on how to address the wearer's current physical state. For instance, if the wearer is having an adverse reaction to a particular pollutant, the system may suggest that the wearer utilize one or more medications, or take other preventative or ameliorating measures to reduce the effects of the adverse reaction. In particular embodiments, the system may also, or alternatively, store the determined environmental pollutant level (e.g., pollutant concentration or exposure level), data regarding the user's physical state, and any related information in computer memory for later analysis.

In various embodiments, while the system is using one or more environmental pollution sensors (e.g., eyewear based sensors) to assess the level of environmental pollutants, the system may also (e.g., at least substantially simultaneously) capture one or more images of a wearer and/or one or more visible pollutants (e.g., smog or UV light radiation) located in close proximity to the wearer (e.g., using a camera, such as a forward or rear facing camera associated with eyewear worn by the wearer). In various embodiments, the system may then analyze the image to further assess the user's exposure and reaction to a particular pollutant (e.g., intense sunlight). The system may then provide one or more suggestions to the wearer as to how to avoid any adverse reaction to the environmental pollutants prior to (or after) the physical manifestation of any symptoms.

Exemplary Technical Platforms

As will be appreciated by one skilled in the relevant field, the present systems and methods may be, for example, embodied as a computer system, a method, or a computer program product. Accordingly, various embodiments may be entirely hardware or a combination of hardware and software. Furthermore, particular embodiments may take the form of a computer program product stored on a computer-readable storage medium having computer-readable instructions (e.g., software) embodied in the storage medium. Various embodiments may also take the form of Internet-implemented computer software. Any suitable computer-readable storage medium may be utilized including, for example, hard disks, compact disks, DVDs, optical storage devices, and/or magnetic storage devices.

Various embodiments are described below with reference to block diagram and flowchart illustrations of methods, apparatuses, (e.g., systems), and computer program products. It should be understood that each block of the block diagrams and flowchart illustrations, and combinations of blocks in the block diagrams and flowchart illustrations, respectively, can be implemented by a computer executing computer program instructions. These computer program instructions may be loaded onto a general purpose computer, a special purpose computer, or other programmable data processing apparatus that can direct a computer or other programmable data processing apparatus to function in a particular manner such that the instructions stored in the computer-readable memory produce an article of manufacture that is configured for implementing the functions specified in the flowchart block or blocks.

The computer instructions may execute entirely on the user's computer, partly on the user's computer, as a stand-alone software package, partly on a user's computer and partly on a remote computer, or entirely on the remote computer or server. In the latter scenario, the remote computer may be connected to the user's computer through any suitable type of network, including but not limited to: a local area network (LAN); a wide area network (WAN), such as the Internet; and/or a cellular network.

These computer program instructions may also be stored in a computer-readable memory that can direct a computer or other programmable data processing apparatus to function in a particular manner such that the instructions stored in the computer-readable memory produce an article of manufacture that is configured for implementing the function specified in the flowchart block or blocks. The computer program instructions may also be loaded onto a computer or other programmable data processing apparatus to cause a series of operational steps to be performed on the computer or other programmable apparatus to produce a computer-implemented process (e.g., method) such that the instructions that execute on the computer or other programmable apparatus provide steps for implementing the functions specified in the flowchart block or blocks.

Example System Architecture

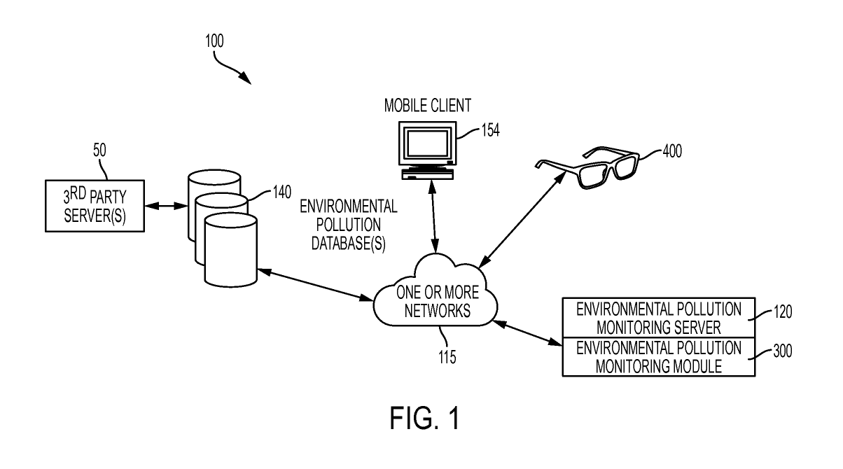

FIG. 1 is a block diagram of an Environmental Pollution Monitoring System 100 according to particular embodiments. As may be understood from this figure, the Environmental Pollution Monitoring System 100 includes One or More Networks 115, One or More Third Party Servers 50, an Environmental Pollution Monitoring Server 120 that may, for example, be adapted to execute an Environmental Pollution Monitoring Module 300, one or more Databases 140 (e.g., one or more Environmental Pollution Databases), One or More Remote Computing Devices 154 (e.g., a smart phone, a tablet computer, a wearable computing device, a laptop computer, a desktop computer, etc.), and One or More Environmental Pollution Monitoring Devices 400, which may, for example, be embodied as eyewear, headwear, clothing, a watch, a hat, a helmet, a cast, an adhesive bandage, a piece of jewelry (e.g., a ring, earring, necklace, bracelet, brooch, etc.), or any other suitable wearable device or other device (e.g., other computing device). In particular embodiments, the one or more computer networks 115 facilitate communication between the One or More Third Party Servers 50, the Environmental Pollution Monitoring Server 120, the one or more Databases 140, the One or More Remote Computing Devices 154, and the one or more Environmental Pollution Monitoring Devices 400.

The one or more networks 115 may include any of a variety of types of wired or wireless computer networks such as the Internet (or other WAN), a private intranet, a mesh network, a public switch telephone network (PSTN), and/or any other type of network (e.g., a network that uses Bluetooth or near field communications to facilitate communication between computing devices). The communication link between the One or More Remote Computing Devices 154 and the Environmental Pollution Monitoring Server 120 may be, for example, implemented via a Local Area Network (LAN) or via the Internet (or other WAN).

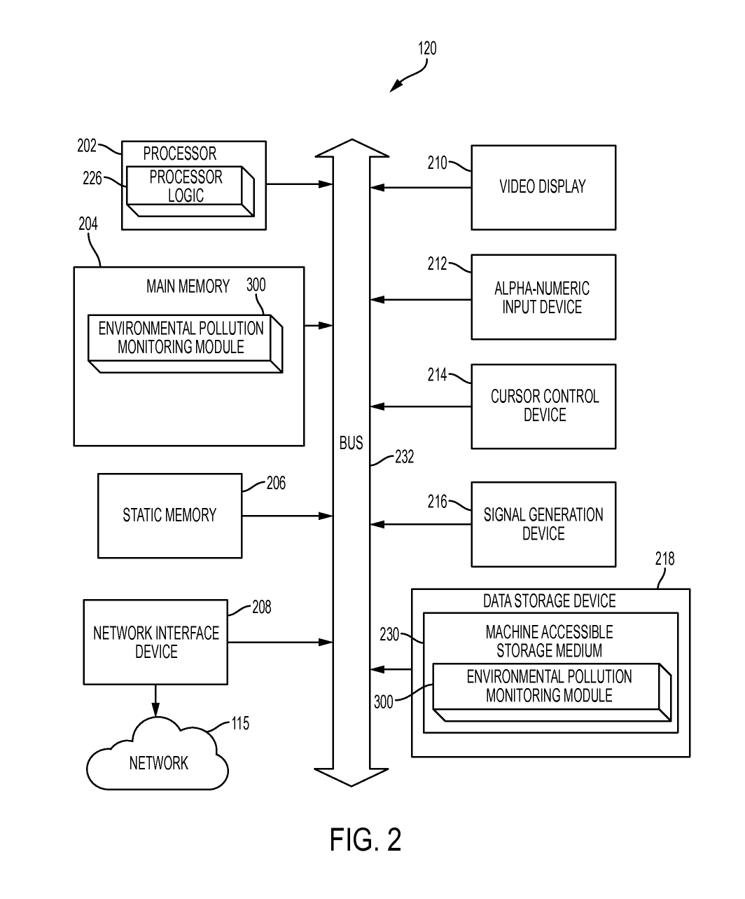

FIG. 2 illustrates a diagrammatic representation of an exemplary architecture of an Environmental Pollution Monitoring Server 120 that may be used within various embodiments of the Environmental Pollution Monitoring System 100. It should be understood that the computer architecture shown in FIG. 2 may also represent the computer architecture for any one of the One or More Remote Computing Devices 154, one or more Third Party Servers 50, and/or the One or More Health Monitoring Devices 400 shown in FIG. 1. In particular embodiments, the Environmental Pollution Monitoring Server 120 may be suitable for use as a computer within the context of the Environmental Pollution Monitoring System 100 that is configured to determine the environmental pollution near a wearer by detecting one or more characteristics of pollution using one or more signals received from one or more sensors coupled to the one or more Health Monitoring Devices 400. In other particular embodiments, the Environmental Pollution Monitoring Device 400 may include an on-board computer processor that is adapted to execute an Environmental Pollution Monitoring Module 300 to determine the environmental pollution near the wearer.

In particular embodiments, the Environmental Pollution Monitoring Server 120 may be connected (e.g., networked) to other computing devices in a LAN, an intranet, an extranet, and/or the Internet as shown in FIG. 1. As noted above, the Environmental Pollution Monitoring Server 120 may operate in the capacity of a server or a client computing device in a client-server network environment, or as a peer computing device in a peer-to-peer (or distributed) network environment. The Environmental Pollution Monitoring Server 120 may be a desktop personal computing device (PC), a tablet PC, a set-top box (STB), a Personal Digital Assistant (PDA), a smartphone, a web appliance, a network router, a switch or bridge, or any other computing device capable of executing a set of instructions (sequential or otherwise) that specify actions to be taken by that computing device. Further, while only a single computing device is illustrated, the term “computing device” shall also be interpreted to include any collection of computing devices that individually or jointly execute a set (or multiple sets) of instructions to perform any one or more of the methodologies discussed herein, or other suitable methodologies.

As shown in FIG. 2, an exemplary Environmental Pollution Monitoring Server 120 includes a processing device 202, a main memory 204 (e.g., read-only memory (ROM), flash memory, dynamic random access memory (DRAM) such as synchronous DRAM (SDRAM) or Rambus DRAM (RDRAM), etc.), a static memory 206 (e.g., flash memory, static random access memory (SRAM), etc.), and a data storage device 218, which communicate with each other via a bus 232.

The processing device 202 represents one or more general-purpose or specific processing devices such as a microprocessor, a central processing unit (CPU), or the like. More particularly, the processing device 202 may be a complex instruction set computing (CISC) microprocessor, reduced instruction set computing (RISC) microprocessor, very long instruction word (VLIW) microprocessor, or processor implementing other instruction sets, or processors implementing a combination of instruction sets. The processing device 202 may also be one or more special-purpose processing devices such as an application specific integrated circuit (ASIC), a field programmable gate array (FPGA), a digital signal processor (DSP), network processor, or the like. The processing device 202 may be configured to execute processing logic 226 for performing various operations and steps discussed herein.

The Environmental Pollution Monitoring Server 120 may further include a network interface device 208. The Environmental Pollution Monitoring Server 120 may also include a video display unit 210 (e.g., a liquid crystal display (LCD) or a cathode ray tube (CRT)), an alpha-numeric input device 212 (e.g., a keyboard), a cursor control device 214 (e.g., a mouse), a signal generation device 216 (e.g., a speaker), and a data storage device 218.

The data storage device 218 may include a non-transitory computing device-accessible storage medium 230 (also known as a non-transitory computing device-readable storage medium, a non-transitory computing device-readable medium, or a non-transitory computer-readable medium) on which is stored one or more sets of instructions (e.g., the Environmental Pollution Monitoring Module 300) embodying any one or more of the methodologies or functions described herein. The one or more sets of instructions may also reside, completely or at least partially, within the main memory 204 and/or within the processing device 202 during execution thereof by the Environmental Pollution Monitoring Server 120—the main memory 204 and the processing device 202 also constituting computing device-accessible storage media. The one or more sets of instructions may further be transmitted or received over a network 115 via a network interface device 208.

While the computing device-accessible storage medium 230 is shown in an exemplary embodiment to be a single medium, the term “computing device-accessible storage medium” should be understood to include a single medium or multiple media (e.g., a centralized or distributed database, and/or associated caches and servers) that store the one or more sets of instructions. The term “computing device-accessible storage medium” should also be understood to include any medium that is capable of storing, encoding, or carrying a set of instructions for execution by the computing device and that causes the computing device to include any one or more of the methodologies of the present invention. The terms “computing device-accessible storage medium” and like terms should accordingly be understood to include, but not be limited to, solid-state memories, optical and magnetic media, etc.

### Exemplary Environmental Pollution Monitoring Device

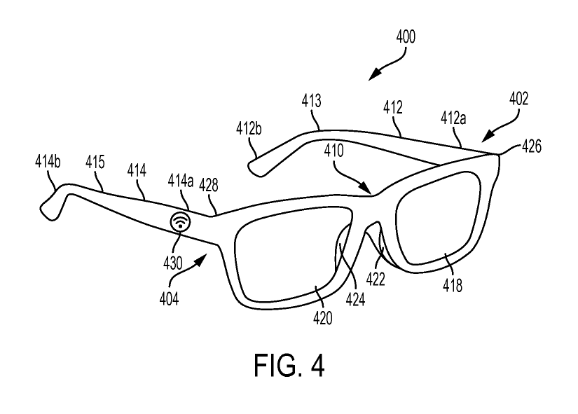

As shown in FIG. 1, the Environmental Pollution Monitoring System 100, in various embodiments, comprises One or More Environmental Pollution Monitoring Devices 400. A particular embodiment of an environmental pollution monitoring device is shown in FIG. 4 as eyewear 400. As shown in this figure, eyewear 400, according to various embodiments, includes: (1) an eyewear frame 410; (2) a first temple 412; and (3) a second temple 414. These various components are discussed in more detail below.

Eyewear Frame

Referring still to FIG. 4, eyewear 400, in various embodiments, includes any suitable eyewear frame 410 that is configured to support one or more lenses 418, 420. In the embodiment shown in this figure, the eyewear frame 410 defines a first end 402 and a second end 404. The eyewear frame 410 may be made of any suitable material such as metal, ceramic, one or more polymers or any combination thereof. In particular embodiments, the eyewear frame 410 is configured to support the first and second lenses 418, 420 about the full perimeter of the first and second lenses 418, 420. In other embodiments, the eyewear frame 410 may be configured to support the first and second lenses 418, 420 about only a portion of each respective lens. In various embodiments, the eyewear frame 410 is configured to support a number of lenses other than two lenses (e.g., a single lens, a plurality of lenses, etc.). In particular embodiments, the lenses 418, 420 may include prescription lenses, sunglass lenses, or any other suitable type of lens (e.g., reading lenses, non-prescription lenses), which may be formed from glass or a suitable polymer.

In various embodiments, the eyewear frame 410 includes a first and second nose pad 422, 424, which may be configured to maintain the eyewear 400 adjacent the front of a wearer's face such that the lenses 418, 420 are positioned substantially in front of the wearer's eyes while the wearer is wearing the eyewear 400. In particular embodiments, the nose pads 422, 424 may comprise a material that is configured to be comfortable when worn by the wearer (e.g., rubber, plastic, etc.). In other embodiments, the nose pads 422, 424 may include any other suitable material (e.g., metal, etc.). In still other embodiments, the nose pads 422, 424 may be integrally formed with the frame 410.

The eyewear frame 410 includes a first and second hinge 426, 428 that attach the first and second temples 412, 414 to the frame first and second ends 402, 404, respectively. In various embodiments, the hinges 426, 428 may be formed by any suitable connection (e.g., tongue and groove, ball and socket, spring hinge, etc.). In particular embodiments, the first hinge 426 may be welded to, or integrally formed with, the frame 410 and the first temple 412, and the second hinge 428 may be welded to, or integrally formed with, the frame 410 and the second temple 414.

First and Second Temples

As shown in FIG. 4, the first temple 412, according to various embodiments, is rotatably connected to the frame 410 at a right angle to extend the first temple 412 substantially perpendicular, substantially parallel, or anywhere in between the right angle to the frame 410. The first temple 412 has a first and second end 412a, 412b. Proximate the first temple second end 412b, the first temple 412 includes an earpiece 413 configured to be supported by a wearer's ear. Similarly, the second temple 414, according to various embodiments, is rotatably connected to the frame 410 at a right angle to extend the second temple 414 substantially perpendicular, substantially parallel, or anywhere in between the right angle to the frame 410. The second temple 414 has a first and second end 414a, 414b. Proximate the second temple second end 414b, the second temple 414 includes an earpiece 415 configured to be supported by a wearer's ear.

Sensors

In various embodiments, one or more sensors 430 may be coupled to the frame 410, the first and second temples 412, 414, the first and second lenses 418, 420, or any other portion of the eyewear 400 in any suitable way. For instance, the one or more sensors 430 may be embedded into the eyewear 400, substantially permanently coupled to the eyewear 400 (e.g. using suitable welding or bonding techniques), and/or detachably coupled to the eyewear 400 (e.g. using a suitable spring-loaded clamp, etc.). In various embodiments, the one or more sensors 430 may be positioned at any point along the eyewear 400. For instance, an air pollution sensor may be disposed adjacent the first temple of the eyewear 400. The one or more sensors 430 may be formed on the inner (back) surface of the frame 410, the first and second temples 412, 414, the first and second lenses 418, 420, or any other portion of the eyewear 400. Also, in some embodiments, the one or more sensors 430 may be formed on the outer (front) surface of the frame 410, the first and second temples 412, 414, the first and second lenses 418, 420, or any other portion of the eyewear 400.

In various embodiments, the one or more sensors 430 that are coupled to the eyewear (or other wearable device) may include one or more environmental pollution sensors that are adapted to determine an individual's exposure to one or more environmental pollutants, and/or one or more health sensors to monitor the wearer's reaction to these pollutants. In various embodiments, the one or more environmental pollution sensors coupled to the eyewear or other environmental pollution monitoring device may include, for example, one or more of the following: (1) one or more ultraviolet light measurement devices (e.g. one or more devices that measure current levels of ultraviolet radiation); (2) one or more light pollutant samplers (e.g., one or more devices that measure current levels of ultraviolet light and blue light); (3) one or more noise pollutant samplers; (4) one or more radioactivity monitors; (5) one or more thermal pollutant samplers; (6) and/or one or more air sampling devices.

In various embodiments, the one or more health sensors may include, for example, one or more heart rate monitors, one or more electrocardiograms (EKGs), one or more electroencephalograms (EEGs), one or more thermometers, one or more transdermal transmitter sensors, one or more blood pressure sensors, one or more pulse oximeters, one or more respiration rate monitors, one or more front-facing cameras, one or more eye-facing cameras, one or more microphones, or any other suitable one or more sensors.

In particular embodiments, the one or more sensors 430 may be detachably or permanently coupled to the eyewear 400 in any suitable way. For example, in various embodiments, the sensors 430 may be embedded into the eyewear 400. In some embodiments, the sensors 430 may be positioned along the brow bar of the eyewear, the one or more of the temples 412, 414 of the eyewear, the lenses of the eyewear 418, 420.

Exemplary System Platform

As noted above, a system, according to various embodiments, is adapted to assess one or more levels of environmental pollution affecting a wearer of a wearable device. Various aspects of the system's functionality may be executed by certain system modules, including the Environmental Pollution Monitoring Module 300. The Environmental Pollution Monitoring Module 300 is discussed in greater detail below.

Environmental Pollution Monitoring Module

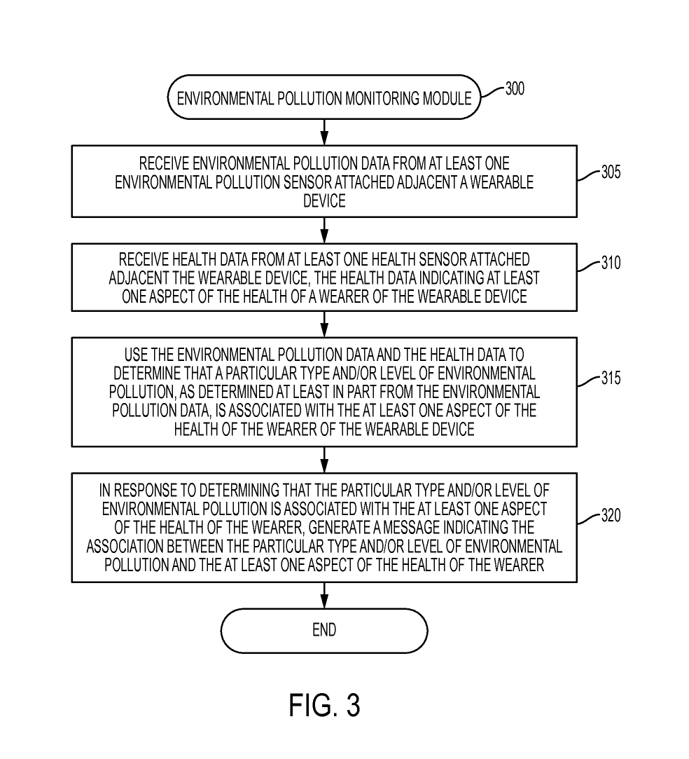

FIG. 3 is a flow chart of operations performed by an exemplary Environmental Pollution Monitoring Module 300, which may, for example, run on the Environmental Pollution Monitoring Server 120, or any other suitable computing device (such as the one or more Environmental Pollution Monitoring Devices 400).

In various embodiments, the system begins, at Step 305 by receiving environmental pollution data from at least one environmental pollution sensor attached adjacent a wearable device. In particular embodiments, the at least one environmental pollution sensor attached to the wearable device (or other environmental pollution monitoring device) may include, for example, one or more of the following: (1) one or more ultraviolet light measurement devices (e.g. one or more devices that measure current levels of ultraviolet radiation); (2) one or more light pollutant samplers (e.g., one or more devices that measure current levels of ultraviolet light and blue light); (3) one or more noise pollutant samplers; (4) one or more radioactivity monitors; (5) one or more thermal pollutant samplers; (6) and/or one or more air sampling devices. In some embodiments, the environmental pollution data may relate to at least one characteristic associated with one or more of the following pollutants: (1) air pollutants (e.g., carbon monoxide, sulfur dioxide, nitrogen oxides, ozone, particulate matter, methane, non-methane hydrocarbons, etc.); (2) light pollutants (e.g., concentration of UV light exposure over time, light trespass, over-illumination, glare, light clutter, skyglow, exposure to blue light, etc.); (3) noise pollutants; (4) radioactive pollutants (e.g., alpha particle emissions, beta particle emissions, gamma ray emissions, etc.); (5) thermal pollutants; and/or (6) any other relevant pollutant.

In particular embodiments, the system may receive the environmental pollution data substantially automatically and/or on a substantially periodic basis (e.g., by the second, by the minute, hourly, daily, etc.). In other embodiments, the system may receive the environmental pollution data at least partially in response to receiving an indication from the wearer that the system should receive the environmental pollution data. For instance, the wearer may speak a voice command to the wearable device requesting that the device take an environmental pollution reading. In various other embodiments, the system may receive the environmental pollution data substantially continuously over a period of time (e.g., throughout the day, during daylight hours, etc.). In yet other embodiments, the system may modify how frequently the system receives the environmental pollution data based at least partially one or more conditions. For example the system may receive information periodically in response to determining that certain environmental conditions are present above a certain level (e.g., UV levels are above a certain threshold). When the system receives information that the UV level exceeds the threshold, the system may monitor the UV level substantially continuously.

In particular embodiments, the system may analyze the received environmental pollution data to determine at least one environmental pollution characteristic associated with the wearer's immediate environment. In various embodiments, the system may analyze the environmental pollution data by comparing various received environmental pollution data to predefined threshold levels. For example, the system may analyze the received environmental pollution data to determine whether there is a concentration of carbon monoxide above a predefined threshold level, a decibel level above a predefined threshold level, a smog level above a predefined threshold level, a blue light level above a predefined threshold level, or UV exposure (e.g., over a particular period of time) above a predefined threshold. In various embodiments, the system may analyze the received environmental pollution data based on an empirical measurement, such as the concentration of particles per million particles of the atmosphere. In various other embodiments, the system may analyze the received environmental pollution data based on a scaled rating. For example, where the system is determining the environmental pollution level of air pollution, the system may determine that the air pollution level is low, moderate, or high.

In various embodiments, the system may chart the analysis of the received environmental pollution data in a visual diagram. For example, the system may chart the changes in the air pollution level in a diagram displayed, for example, on the lens of the eyewear or on a separate display screen associated with the wearer. In various other embodiments, the system may chart the analysis of the received environmental pollution data substantially continuously.

In various embodiments, the system may analyze a particular received environmental pollution data at a particular time of day (e.g., morning, noon, night, etc.) and/or when the wearer arrives at a particular location (e.g., outdoors, in the city center, etc.). In various other embodiments, the system may analyze the environmental pollution data automatically or in response to receiving manual requests from the wearer. For example, the system may receive a voice command from the wearer requesting that the system determine the environmental pollution level around the wearer at that time.

At Step 310, the system is configured to receive health data from at least one health sensor attached adjacent the wearable device, the health data indicating at least one aspect of health of a wearer of the wearable device. In various embodiments, the health data may relate to at least one health characteristic associated with the wearer, and may include, for example: (1) the wearer's heart rate; (2) the wearer's heart rhythm; (3) the wearer's body temperature; (5) one or more images of the wearer or the wearer's immediate environment; (6) one or more sounds generated by the wearer's body (e.g., pulse) or the wearer's immediate environment; (7) the wearer's blood pressure; (8) the wearer's oxygen saturation level; (10) the wearer's pupil size; (11) the wearer's respiration rate; (12) one or more characteristics of the wearer's skin (e.g., paleness or redness); and/or (13) any other suitable attribute of the wearer or the wearer's environment.

In some embodiments, the system may receive the health data, for example, as an image or plurality of images captured by the eyewear. In particular embodiments, the system may receive such an image from an eye-facing camera associated with the wearable device. In some embodiments, the image captured by the wearable device may be an infrared image. In other embodiments, the system may receive the health data from the Environmental Pollution Database 140 to obtain a wearer's known sensitivities and/or medical conditions (e.g., asthma or known allergies).

In various embodiments, the system may capture the above-referenced health data substantially automatically and/or on a substantially periodic basis (e.g., by the second, by the minute, hourly, daily, etc.). In other embodiments, the system may receive the health data at least partially in response to receiving an indication from the wearer that the system should receive the health data. For instance, the wearer may speak a voice command to the wearable device requesting that the device take a heart rate reading. In various other embodiments, the system may receive the health data substantially continuously over a period of time (e.g., throughout the day, during daylight hours, etc.). In various embodiments, the system may store the health data in local or remote memory.

In particular embodiments, the system may receive a request from the wearer to have the health data received from the at least one health sensor at least partially in response to receiving environmental pollution data from the at least one environmental pollution sensor. For example, when the system receives environmental pollution data that indicates an environmental pollution sensor detects a certain air pollutant, the system may, at least partially in response to receiving the air pollution data, obtain a reading of the user's respiration rate. As a further example, the system may receive environmental pollution data from a UV sensor that indicates an environmental pollution sensor detects a certain level of UV light above a pre-determined threshold. At least partially in response to the UV light being above a pre-determined threshold, the system may receive health data from an eye-facing camera associated with the wearable device to help detect relevant changes in the wearer's skin (e.g., rashes or sunburn).

At Step 315, the system uses the environmental pollution data and the health data to determine that a particular type and/or level of environmental pollution, as determined at least in part from the environmental pollution data, is associated with the at least one aspect of the wearer's health of the wearable device. In particular embodiments, the system may use the environmental pollution data and the health data to determine that a particular type and/or level of environmental pollution is associated with at least one physical manifestation of an adverse reaction to an environmental pollutant (e.g., a respiratory rate, a heart rate, a skin condition, eye strain, disrupted circadian rhythm, or a brainwave activity), at least one geographical location (e.g., GPS coordinates), and/or at least one time of day. In some embodiments, the system may determine that a particular type and/or level of environmental pollution is associated with both a geographic location and a specific time, with both a physical manifestation and a pollution rating, or any other combination of factors described herein.

In various embodiments, the system may determine that a particular type and/or level of environmental pollution, as determined at least in part from the environmental pollution data, is associated with at least one pollution characteristic. For example, an increase in respiratory rate may show a correlation with an increase in the level of sulfur dioxides. It should be understood from this disclosure that the system may determine that the environmental pollution associated with the aspect of the wearer's health is a combination of characteristics that allow the system to determine the effect of the multiple types and/or levels of environmental pollution on the wearer.

In various embodiments, the system stores, in memory, the determination that a particular type and/or level of environmental pollution is associated with the at least one aspect of the health of the wearer. In various embodiments, the system may store the association between the environmental pollution level and the aspect of the wearer's health substantially automatically and/or after (e.g., in response to) determining that a particular type and/or level of environmental pollution is associated with the at least one aspect of the wearer's health. In other embodiments, the system may store the association between the environmental pollution level and the aspect of the wearer's health after (e.g., at least partially in response to) receiving manual input from the wearer. In various embodiments, the system may store the association between the environmental pollution level and the aspect of the wearer's health in a suitable Database 140.

At Step 320, in response to determining that the particular type and/or level of environmental pollution is associated with the at least one aspect of the wearer's health, the system generates a message indicating the association between the particular type and/or level of environmental pollution and the at least one aspect of the wearer's health. In various embodiments, the system may generate a message to the wearer or other suitable individual of the association between the particular type and/or level of environmental pollution and the at least one aspect of the health of the wearer. For example, in some embodiments, the system may notify the wearer and/or the wearer's physician of the association of a particular environmental pollution to a particular aspect of the wearer's health (e.g., the system may notify the wearer that they appear to have an allergy to ragweed). In various embodiments, the system pools data from multiple wearers within a geographic area and may inform health officials regarding geographically larger environmental pollution trends.

In particular embodiments, the system may generate a message to the wearer of the association between the particular type and/or level of environmental pollution and the at least one aspect of the wearer's health, for example, by displaying an image on the lens of the eyewear 400, or a display screen associated with the eyewear 400, or by communicating the association through an audio speaker to the wearer. In some embodiments, the system may generate a message to the wearer of the association by sending a notification to the wearer's mobile device. In particular embodiments, the system may generate a message via an electronic communication such as email or SMS. In certain embodiments, the system may generate a message of all associations made over a period of time (e.g., a day, week, month, etc.).

In other embodiments, the system may generate a message of a particular association between the particular type and/or level of environmental pollution and the at least one aspect of the health of the wearer after (e.g., at least partially in response to) detecting a particular event. For example, the system may generate a message of the association in response to the system ceasing to detect the physical manifestation of an adverse reaction to an environmental pollutant and/or at least partially in response to the system detecting a decrease below a predetermined threshold level of an environmental pollutant. In other embodiments, the system may generate a message of the association based on historical data or the data of other users. For example, the system may have made an association between particular environmental pollution data (e.g., high smog levels) and particular health data (e.g., increased wearer heart rate) at a given time or geographical location in the past, and may alert the wearer when the wearer approaches a given geographical location where the environmental pollution at issue has been reported above a threshold level by other wearers. In still other embodiments, the system may generate a message at a certain time of day or at a certain geographical location. As an example, the system may generate a message to the wearer of an association between the environmental pollution and an aspect of the wearer's health at the end of the day or when the user is approaching, or en route to, a certain geographical location that is associated with environmental pollution factors that the system has determined may adversely impact the wearer's health.

In various embodiments, the system may provide the wearer with one or more suggested actions to address or ameliorate the effects of environmental pollution (e.g., that the system has determined adversely impacts the wearer's health). In various embodiments, the system may provide one or more suggested actions to the wearer in any suitable way. For example, the system may provide one or more suggested actions to the wearer by displaying an image on the lens of the eyewear, or on a display screen associated with the wearer, or through an audio speaker. In some embodiments, the system provides one or more suggested actions to the wearer by sending a notification to the wearer's mobile by email, or by SMS.

In various embodiments, the one or more suggested actions to address the environmental pollution may include one or more preventative health measures. In particular embodiments, the one or more suggested actions may include, for example: (1) wearing an anti-pollution mask; (2) applying sunscreen to the wearer's skin; (3) utilizing ear protection; (4) wearing protective lenses; (5) abstaining from vigorous physical activity during a certain time period; (6) taking an alternate route to a destination; and/or (7) utilizing a suitable medication to address the environmental pollution. In other embodiments, the one or more suggested actions to address the environmental pollution level may include suggesting that the wearer visit one or more websites containing information on preventative health measures or environmental pollution. As another example, where the environmental pollution is at an elevated state, the system may suggest a nearby location where preventative health products may be purchased to aid in ameliorating adverse effects of environmental pollution.

In various embodiments, the system may provide one or more suggested actions to the wearer to address or ameliorate the effects of environmental pollution immediately after the system notifies the wearer of the association between the wearer's physical condition and the environmental pollution level. In other embodiments, the system may provide one or more suggested actions to the wearer after (e.g., in response to) detecting a particular environmental condition. For example, the system may provide one or more suggested actions to the wearer after the system detects a change in the level of a particular environmental pollutant. In some embodiments, the system may provide one or more suggested actions to the wearer within a particular period of time after the system notifies the wearer of the association between the wearer's physical condition and the environmental pollution level. For instance, the system may provide one or more suggested actions to the wearer one hour after the system associates a physical condition of the wearer (e.g. wheezing) with an environmental pollutant (e.g. cigarette smoke). In still other embodiments, the system may provide one or more suggested actions to the wearer at a particular time of day or when the wearer reaches a particular geographical area.

In various embodiments, the system, when executing the Environmental Pollution Monitoring Module 300, may omit particular steps, perform particular steps in an order other than the order presented above, or perform additional steps not discussed directly above. It should also be understood that various steps executed “in response to” a particular event occurring, or a particular condition being satisfied, may also be executed “at least partially in response to” the particular event occurring or the particular condition being satisfied.

Exemplary User Experience

Individual Tracking

In a particular example of using the Environmental Pollution Monitoring Module 300 to monitor environmental pollution, the wearer may put on the one or more Environmental Pollution Monitoring Devices 400 in the morning and continue to wear the device throughout the day. During this time, the system may, for example, track air pollution, including sulfur dioxide, nitrogen oxides, ozone, particulate matter, carbon monoxide, hydrocarbons, and/or volatile organic compounds using an air pollution sensor associated with the one or more Environmental Pollution Monitoring Devices 400. The system may then use a GPS device associated with the one or more Environmental Pollution Monitoring Devices 400 or other device associated with the wearer (e.g., the user's smartphone) to track the location of the wearer contemporaneously with the concentration of soot particulates and sulfur dioxide levels measured by the air pollution sensor. The system may use this information to determine the relative localized level of environmental pollution around the wearer.

The system may store the environmental pollution levels and chart the highs and lows of various pollutants over the course of a week or other predetermined time period, to determine the times and days when the user experiences the highest and lowest pollution levels. For example, in geographical locations where the environmental pollution levels are relatively high from 6:00 a.m. to 9:00 a.m. and from 4:00 p.m. to 7:00 p.m., the system may notify the wearer that the highest pollution levels relative to the wearer's geographic locations during those times occurs from 6:00 a.m. to 9:00 a.m. and from 4:00 p.m. to 7:00 p.m. The wearer may then use this information to alter the wearer's activities so that the wearer can avoid the highest peaks of environmental pollution.

Predictive Modeling

Just as the system may track environmental pollution relative to the wearer over a period of time, the system, in a particular example, may also predictively model environmental pollution levels based on historical data other than time. The system may generate this model, for example, by tracking the environmental pollution levels relative to the wearer, in combination with some other data, for example, weather data, traffic data, event data, meteorological data, etc. The system may then generate a predictive model that will enable the wearer to take proactive, rather than reactive measures to ameliorate the effects of environmental pollution. For instance, the system may use historic data regarding the time and locations of travel with live traffic data to generate an estimation of environmental pollution over a given route when certain weather events occur. The wearer may use this information to plan their travels accordingly.

Regional Modeling

In a further particular example of the Environmental Pollution Monitoring Module 300 of the One or More Wearable Environmental Pollution Monitoring Devices 400 monitoring environmental pollution levels, the system may track multiple wearers within a geographical location to provide an accurate measure of the environmental pollution over a larger area. The system may track multiple wearers by storing the environmental pollution data for multiple wearers and analyzing the data to calculate the environmental pollution level over the area the data covers.

For example, if multiple wearers encounter a particular environmental pollution in a particular area that is distinct from the environmental pollution a particular wearer normally encounters, the system may provide the particular wearer with a notification that the environmental pollution in the particular area is different from the environmental pollution the wearer normally encounters. For instance, if a particular wearer travels a different route or travels to a different location from their normal routine, the system may alert the wearer of particular environmental pollutants prior to the wearer arriving in that particular location. The system may also advise the wearer to take alternate routes in order to avoid the different environmental pollution that the wearer does not normally encounter.

Health Monitoring

In a further particular example of using the Environmental Pollution Monitoring Module 300 of the One or More Wearable Environmental Pollution Monitoring Devices 400 to monitor environmental pollution levels, the system tracks the wearer's response to various environmental pollutants throughout the day via the wearable device. The system tracks the wearer's reaction to various environmental pollutants by monitoring the wearer's pupil size, heart rate, respiration rate, skin condition (e.g., skin color, hives, rashes), etc. The system may also monitor changes in the wearer's pupil size, heart rate, perspiration level, respiration rate, skin condition, or other health data using an eye-facing camera.

For example, if the wearer has an abnormal increase in their respiratory rate simultaneously with an abnormal environmental pollution level, the system may alert the wearer or other individual (e.g., the wearer's physician) to the predicted sensitivity. The system may then provide instructions to the wearer suggesting the wearer take measures to ameliorate their condition and suggest other ways to avoid exposure to the pollutant in the future. The system may, for example, provide these suggestions to the wearer through a suitable notification sent to the wearer's mobile device.

### CONCLUSION

Many modifications and other embodiments of the invention will come to mind to one skilled in the art to which this invention pertains, having the benefit of the teaching presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for the purposes of limitation.

What is claimed is:

1. A computer-readable medium storing computer-executable instructions for:
receiving environmental pollution data from at least one environmental pollution sensor attached adjacent a wearable device;
receiving health data from at least one health sensor attached adjacent the wearable device, the health data indicating at least one aspect of health of a wearer of the wearable device;
using the environmental pollution data and the health data to determine that a particular type and/or level of environmental pollution, as determined at least in part from the environmental pollution data, is associated with the at least one aspect of the wearer's health; and
in response to determining that the particular type and/or level of environmental pollution is associated with the at least one aspect of the wearer's health, generating a message indicating the association between the particular type and/or level of environmental pollution and the at least one aspect of the wearer's health.

2. The computer-readable medium of claim 1, wherein the wearable device comprises a pair of eyewear.

3. The computer-readable medium of claim 2, wherein the at least one health sensor comprises a camera that is positioned to capture an image of at least a portion of the wearer's body when the wearer is wearing the wearable device.

4. The computer-readable medium of claim 3, wherein:
the at least one environmental pollution sensor comprises a UV sensor; and
the aspect of the wearer's health is a presence of a sunburn on the wearer's skin.

5. The computer-readable medium of claim 4, wherein:
the environmental data comprises UV data received from the UV sensor; and
the computer-readable medium further stores computer-executable instructions for:
determining, based at least in part on the UV data and the image, an amount of UV exposure the wearer's skin can tolerate before becoming sunburned.

6. The computer-readable medium of claim 3, wherein:
the at least one environmental pollution sensor comprises a sensor for sensing a presence of at least one airborne allergen; and
the aspect of the wearer's health is a respiratory ailment.

7. The computer-readable medium of claim 6, wherein:
the at least one airborne allergen comprises smog; and
the aspect of the wearer's health is asthma.

8. The computer-readable medium of claim 2, wherein the at least one health sensor comprises an oxygen saturation monitor.

9. The computer-readable medium of claim 6, wherein:
the at least one airborne allergen comprises an allergen; and
the aspect of the wearer's health is one or more respiratory issues.

10. The computer-readable medium of claim 7, wherein the at least one airborne allergen comprises smog.

11. The computer-readable medium of claim 2, wherein the at least one health sensor is embedded within the pair of eyewear.

12. A computer-readable medium comprising computer-executable instructions for:
receiving environmental pollution data from a plurality of environmental sensors, each of the sensors being attached adjacent a respective particular wearable device that is being worn by a respective particular user while the user is located within a particular geographical zone;
using the pollution data from each of the plurality of environmental sensors to determine a respective level of a particular pollutant at a particular respective location within the particular geographical zone;
determining, at least in part on the determined respective levels of the particular pollutant at particular respective locations within the particular geographical zone, an approximate aggregate level of the particular pollutant within the particular geographical zone.

13. The computer-readable medium of claim 12, wherein the wearable device comprises a pair of eyewear.

14. The computer-readable medium of claim 13, wherein the plurality of environmental sensors comprises UV sensors.

15. The computer-readable medium of claim 13, wherein the plurality of environmental sensors comprises smog sensors.

16. A computer-implemented method of monitoring environmental pollution adjacent
a wearable device, the method comprising:
receiving, by at least one processor, environmental pollution data from at least one environmental pollution sensor attached adjacent the wearable device;
receiving, by at least one processor, health data from at least one health sensor attached adjacent the wearable device, the health data indicating at least one aspect of health of a wearer of the wearable device;
using, by at least one processor, the environmental pollution data and the health data to determine that a particular type and/or level of environmental pollution, as determined at least in part from the environmental pollution data, is associated with the at least one aspect of the wearer's health; and
in response to determining that the particular type and/or level of environmental pollution is associated with the at least one aspect of the wearer's health, generating, by at least one processor, a message indicating the association between the particular type and/or level of environmental pollution and the at least one aspect of the wearer's health.

17. The computer-implemented method of claim 16, wherein the wearable device comprises a pair of eyewear.

18. The computer-implemented method of claim 16, wherein:
the at least one environmental pollution sensor comprises a UV sensor; and
the aspect of the wearer's health is a presence of a sunburn on the wearer's skin.

19. The computer-implemented method of claim 16, wherein:
the at least one environmental pollution sensor comprises a sensor for sensing a presence of at least one airborne allergen; and
the aspect of the wearer's health is a respiratory ailment.

20. The computer-implemented method of claim 19, wherein:
the at least one airborne allergen comprises smog; and
the aspect of the wearer's health is asthma.
